(19) United States
(12) Patent Application Publication
(10) Pub. No.: US 2014/0213903 A1
(43) Pub. Date: Jul. 31, 2014

(54) METHOD AND APPARATUS FOR PROVIDING ULTRASOUND TREATMENT

(71) Applicant: Samsung Electronics Co., Ltd., Suwon-si (KR)

(72) Inventors: Joon-ho SEO, Seoul (KR); Won-chul BANG, Seongnam-si (KR)

(73) Assignee: Samsung Electronics Co., Ltd., Suwon-si (KR)

(21) Appl. No.: 14/102,844

(22) Filed: Dec. 11, 2013

(30) Foreign Application Priority Data
Jan. 25, 2013 (KR) 10-2013-0008742

(51) Int. Cl. A61N 7/02; A61B 8/08; A61B 8/00; A61B 19/00; A61B 8/14

(52) U.S. Cl. CPC A61N 7/02; A61B 19/5212; A61B 19/5202; A61B 8/14; A61B 8/4444; A61B 8/461; A61B 8/5207
USPC 600/439; 601/3

(57) ABSTRACT

An apparatus configured to provide ultrasound treatment, includes an applicator configured to radiate ultrasound to a subject. The apparatus further includes an image capturing unit configured to capture an image of a coupling guiding line on a patient where the applicator is to be placed, and a computer configured to set a target location of the applicator based on the image and a current location of the applicator. The apparatus further includes a location adjusting unit configured to adjust a location of the applicator, and a control unit configured to control the location adjusting unit to adjust the location of the applicator to the target location. The applicator is configured to radiate ultrasound at the adjusted location.

### CROSS-REFERENCE TO RELATED APPLICATIONS

This application claims the benefit under 35 USC 119(a) of Korean Patent Application No. 10-2013-0008742, filed on Jan. 25, 2013, in the Korean Intellectual Property Office, the entire disclosure of which is incorporated herein by reference for all purposes.

### BACKGROUND

1. Field

The following description relates to a method and an apparatus for providing ultrasound treatment.

2. Description of Related Art

Recent developments of medical technologies have lead to the use of non-invasive surgery for the local treatments of tumors, instead of minimally invasive surgery. High-intensity focused ultrasound (HIFU) treatment is a non-invasive surgery technique widely used to treat tumors due to its main advantage of being unharmful to the human. During HIFU treatment, tissues including the tumor are necrotized by focusing and radiating high-intensity ultrasound on the tumor. That is, when a patient requires a tumor treatment, an ultrasound treatment and diagnosis system is used to radiate ultrasound on the tumor through an ultrasound treatment apparatus, and whether the treatment has been completed is determined by acquiring ultrasound images of tissues including the tumor through an ultrasound diagnostic apparatus.

During the ultrasound treatment, an applicator of the ultrasound treatment apparatus needs to be placed on patient's skin for a long time. However, in the case of HIFU treatment, as a transducer that generates and radiates high-intensity continuous ultrasound is heated, patient's skin in contact with an applicator of an ultrasound treatment apparatus is also heated. Thus, it is difficult to continuously maintain the applicator of the ultrasound treatment apparatus on the patient's skin, and the application should be separated from the patient's skin repeatedly. Accordingly, there is a need for a method and a system for providing ultrasound treatment whereby an ultrasound applicator may be continuously maintained in contact with the same part of the patient's skin.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, an apparatus configured to provide ultrasound treatment, includes an applicator configured to radiate ultrasound to a subject. The apparatus further includes an image capturing unit configured to capture an image of a coupling guiding line on a patient where the applicator is to be placed, and a computer configured to set a target location of the applicator based on the image and a current location of the applicator. The apparatus further includes a location adjusting unit configured to adjust a location of the applicator, and a control unit configured to control the location adjusting unit to adjust the location of the applicator to the target location. The applicator is configured to radiate ultrasound at the adjusted location.

The image capturing unit may be installed in the applicator.

The image capturing unit may include a light-emitting device.

The computer may be configured to calculate a distance from a center of the coupling guiding line in the image to a center of the applicator, and an angle between a line perpendicular to the coupling guiding line at the center thereof and a line perpendicular to the applicator at the center thereof. The computer may be further configured to set the target location based on the distance and the angle.

The apparatus may further include a diagnostic probe installed in the applicator, and configured to radiate ultrasound to a tissue including the subject at the adjusted location, and receive an echo signal reflected from the tissue.

The computer may be further configured to produce an ultrasound image of the tissue based on the echo signal, and obtain a fine adjustment of the adjusted location based on a pre-obtained diagnostic image of the tissue including the subject and the ultrasound image.

The control unit may be further configured to control the location adjusting unit to adjust the adjusted location based on the fine adjustment.

The apparatus may further include a display device configured to display the image of the coupling guiding line and/or the ultrasound image.

The computer may be configured to determine whether the applicator is within a predetermined distance from the coupling guiding line based on the adjusted location and the coupling guiding line imaged at the adjusted location. The control unit may be configured to control the applicator to radiate the ultrasound at the adjusted location in response to the applicator being determined to be within the predetermined distance.

The computer may be further configured to re-determine whether the applicator is within the predetermined distance from the coupling guiding line based on the adjusted location and the coupling guiding line imaged at the adjusted location, in response to the applicator being controlled to radiate the ultrasound. The computer may be further configured to determine whether the ultrasound treatment is completed in response to the applicator being re-determined to be within the predetermined distance. The control unit may be further configured to re-control the applicator to radiate the ultrasound at the adjusted location in response to the ultrasound treatment being determined to be not completed.

The control unit may be further configured to control the image capturing unit, the computer, and the location adjusting unit, to locate the applicator at the coupling guiding line, in response to the applicator being determined or re-determined to be not within the predetermined distance.

In another aspect, a method of providing ultrasound treatment, the method includes capturing an image of a coupling guiding line on a patient where an applicator is to be placed, the applicator configured to radiate ultrasound to a subject. The method further includes setting a target location of the applicator based on the image and a current location of the applicator, adjusting a location of the applicator to the target location, and radiating the ultrasound at the adjusted location.

The setting of the target location may include calculating a distance from a center of the coupling guiding line in the image to a center of the applicator, and an angle between a line perpendicular to the coupling guiding line at the center thereof and a line perpendicular to the applicator at the center thereof. The setting of the target location may further include setting the target location based on the distance and the angle.

The method may further include radiating ultrasound to a tissue including the subject at the adjusted location, receiving an echo signal reflected from the tissue, and producing an ultrasound image of the tissue based on the echo signal. The method may further include obtaining a fine adjustment of the adjusted location based on a pre-obtained diagnostic image of the tissue including the subject and the ultrasound image, and adjusting the adjusted location based on the fine adjustment.

The radiating of the ultrasound may include determining whether the applicator is within a predetermined distance from the coupling guiding line based on the adjusted location and the coupling guiding line imaged at the adjusted location. The radiating of the ultrasound may further include radiating the ultrasound at the adjusted location in response to the applicator being determined to be within the predetermined distance.

The capturing of the image, the setting of the target location, and the adjusting of the location of the applicator may be repeated, in response to the applicator being determined to be not within the predetermined distance.

The method may further include determining whether the applicator is within a predetermined distance from the coupling guiding line based on the adjusted location and the coupling guiding line imaged at the adjusted location, in response to the radiating of the ultrasound. The method may further include determining whether the ultrasound treatment is completed in response to the applicator being determined to be within the predetermined distance, and re-radiating the ultrasound at the adjusted location in response to the ultrasound treatment being determined to be not completed.

The capturing of the image, the setting of the target location, and the adjusting of the location of the applicator may be repeated, in response to the applicator being determined to be not within the predetermined distance.

The coupling guiding line may have a color that is different from a color of skin of the patient, and may be marked on the skin.

A non-transitory computer-readable storage medium storing a program including instructions may cause a computer to execute the method.

In still another general aspect, an apparatus includes an applicator configured to radiate ultrasound to a patient, an image capturing unit configured to capture an image of a mark on the patient where the applicator is to be placed, and a computer configured to set a target location of the applicator based on the image.

The apparatus may further include a location adjusting unit configured to adjust a location of the applicator. The computer may be further configured to control the location adjusting unit to adjust the location of the applicator to the target location, and control the applicator to radiate ultrasound at the adjusted location.

Other features and aspects will be apparent from the following detailed description, the drawings, and the claims.

### BRIEF DESCRIPTION OF THE DRAWINGS

FIG. 1 is a diagram illustrating an example of an apparatus for providing ultrasound treatment.

FIG. 2 is a diagram illustrating an example of an unit for providing ultrasound treatment in the system of FIG. 1.

FIG. 3 is a view illustrating an example of an applicator including an image capturing unit of the unit of FIG. 2.

FIGS. 4A and 4B are views illustrating examples of the applicator including the image capturing unit of the unit of FIG. 2.

FIG. 5 is a perspective view illustrating another example of the applicator including the image capturing unit of the unit of FIG. 2.

FIG. 6 is a perspective view of a diagnostic probe and an image capturing unit installed in the applicator of FIG. 5.

FIG. 7 is a flowchart illustrating an example of a method of providing ultrasound treatment.

FIG. 8 is a flowchart illustrating an example of an operation of adjusting a location of an applicator.

FIG. 9 is a flowchart illustrating an example of an operation of radiating ultrasound from an applicator at an adjusted location.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

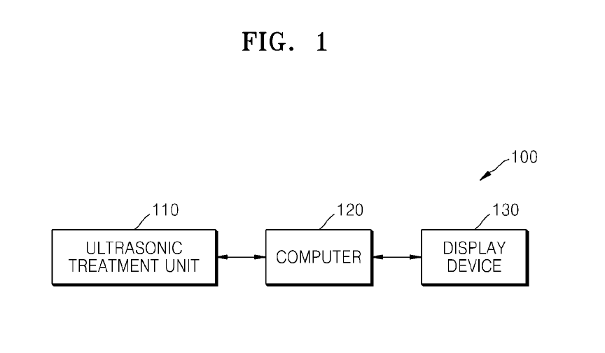

FIG. 1 is a diagram illustrating an example of an ultrasound treatment apparatus 100. Referring to FIG. 1, the ultrasound treatment apparatus 100 includes an ultrasound treatment unit 110, a computer 120, and a display device 130. Other components than those shown in FIG. 1 may be additionally included in the ultrasound treatment apparatus 100. In addition, unlike in FIG. 1, the components of the ultrasound treatment apparatus 100 may not be physically separated from each other, but may be integrated with each other.

If a lesion, such as a tumor, appears on a body tissue, the ultrasound treatment apparatus 100 is used to radiate ultrasound to the lesion to cause necrosis thereof, and subsequently, receives an echo signal from the tissue including the lesion. Then, the ultrasound treatment apparatus 100 produces and displays images of the tissue including the lesion to help a medical practitioner determine whether the lesion has disappeared or treatment is still needed.

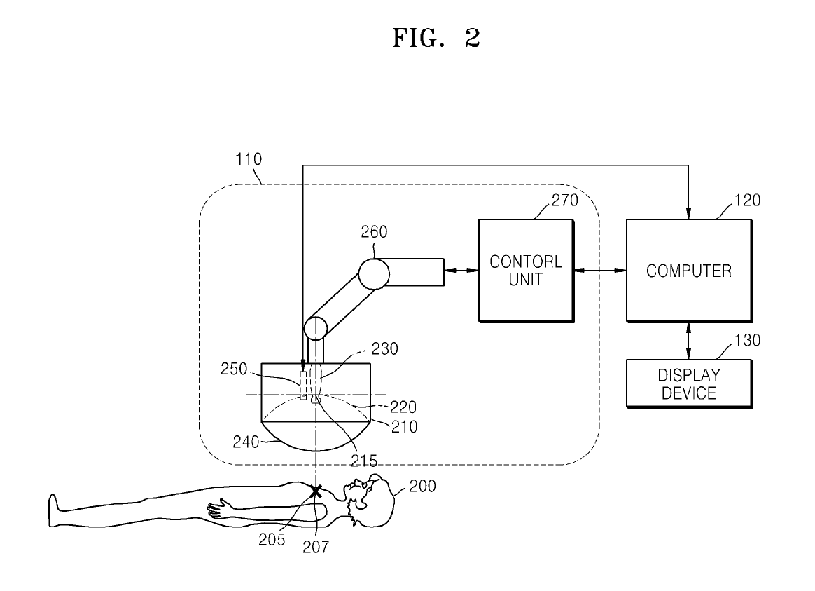

The ultrasound treatment unit 110 radiates ultrasound to a subject, such as a lesion, to necrotize the lesion for treatment. The ultrasound radiated by the ultrasound treatment unit 110 may be high intensity focused ultrasound (HIFU). In more detail, the ultrasound treatment unit 110 includes a diagnostic probe that radiates ultrasound, and the diagnostic probe may be integrated with an applicator, or may be separately configured. The diagnostic probe radiates ultrasound to a tissue including a subject, such as lesion, and receives an echo signal from the tissue including the subject. The echo signal may be used to monitor, for example, a change in temperature of the tissue including the subject, as well as to produce an ultrasound image. Operations and functions of the ultrasound treatment apparatus of FIG. 1 will be described in detail below in connection with FIG. 2.

The computer 120 receives a command from a user, calculates information to be used to control the ultrasound treatment unit 110, produces a signal to control the ultrasound treatment unit 110, and produces an image based on data received from the ultrasound treatment unit 110. For example, the computer 120 produces an ultrasound image of a tissue including a subject, such as lesion, based on an echo signal received from the diagnostic probe of the ultrasound treatment unit 110.

The display device 130 receives a signal to display an image produced by the computer 120, and displays an ultrasound image on a display unit based on the received signal.

FIG. 2 is a diagram illustrating an example of the ultrasound treatment unit 110 in the ultrasound treatment apparatus 100 of FIG. 1. Referring to FIG. 2, the ultrasound treatment unit 110 includes an applicator 210, a location adjusting unit 260, and a control unit 270. The applicator 210 includes a transducer 220, a diagnostic probe 230, a membrane 240, and an image capturing unit 250. One of ordinary skill in the art can understand that other components in addition to those shown in FIG. 2 may be included in the ultrasound treatment unit 110.

The transducer 220 generates ultrasound, and radiates the ultrasound to a subject. The subject may be a lesion in a body of a patient 200. The transducer 220 includes an ultrasound piezo resonator that converts electric energy into ultrasound or vice versa. The transducer 220 may include a plurality of ultrasound piezo resonators, which may be arranged in an array, an n×m matrix, or a circle. If HIFU is radiated, the transducer 220 may have an arch shape or a concave dish shape in order to concentrate the radiated ultrasound. In addition, the transducer 220 may include several separate transducers that radiate ultrasound to one point. The transducer 220 is installed in a housing of the applicator 210. A cone-beam type ultrasound beam may be formed along a path of ultrasound radiated from the transducer 220.

The diagnostic probe 230 radiates ultrasound to a tissue including a subject, and receives an echo signal reflected from the tissue. Although the diagnostic probe 230 may be separated from the applicator 210, the diagnostic probe 230 may be installed in the applicator 210 as shown in FIG. 2. Thus, if the diagnostic probe 230 is installed in the applicator 210, and the applicator 210 is placed in contact to any part of skin (or surface) of the patient 200, the diagnostic probe 230 radiates ultrasound to a tissue including a subject, receives an echo signal reflected from the tissue, and transmits the received echo signal to the control unit 270.

The membrane 240 is located in an area where ultrasound from the transducer 220 is radiated, and forms, with the transducer 220, a space in which a cooling fluid circulates. The membrane 240 is a film through which the cooling fluid does not pass, and may be formed of an elastic material. When the membrane 240 is formed of the elastic material, it is possible to increase a coupling area of the membrane 240 to skin of the patient 200. A side of the membrane 240 is coupled to the skin of the patient 200, and another side thereof contacts the cooling fluid.

The image capturing unit 250 captures an image of a coupling guiding line 205 that represents a coupling region on skin of the patient 200 where the applicator 210 is placed. The coupling guiding line 205 has a different color from a color of the skin of the patient 200, and denotes marking information marked on the skin of the patient 200. After a medical practitioner identifies a location of a subject, such as lesion, in the body of the patient 200, the coupling guiding line 205 is marked on a proper part of the skin of the patient 200 before providing ultrasound treatment. For example, the coupling guiding line 205 may be marked on any part of the skin of the patient 200 that is closest to the subject, considering a depth to which ultrasound may be radiated and a location of the subject. The coupling guiding line 205 may be marked in a triangular, quadrilateral, or circular shape, may be marked as a plurality of dots, or may be marked by ‘X’, using lines as shown in FIG. 2. The coupling guiding line 205 may be marked so that a normal direction of a skin surface of the patient 200 may be identified and that the applicator 210 is perpendicularly placed on the skin surface at a center 207 of the coupling guiding line 205. In this example, the coupling guiding line 205 may have a symmetrical structure.

The image capturing unit 250 captures an image of skin of the patient 200 with the coupling guiding line 205, and transmits data of the image to the computer 120. The image capturing unit 250 and the computer 120 may be connected to each other through an interface, such as a universal serial bus (USB). If a user of the ultrasound treatment apparatus 100 enters an image capturing command, the image capturing unit 250 may continuously capture images in real time. The image capturing unit 250 is installed in the applicator 210, and may include a light-emitting device. For example, the light-emitting device may be a light-emitting diode (LED). The image capturing unit 250 and the light-emitting device may have a subminiature size. The light-emitting device may radiate light according to an operation of the image capturing unit 250. For example, when the image capturing unit 250 captures images of the skin of the patient 200 with the coupling guiding line 205 according to control of the control unit 270, the light-emitting device may radiate light so that the image capturing unit 250 may capture high luminescence images.

The location adjusting unit 260 adjusts a location of the applicator 210. The location adjusting unit 260 is controlled by the control unit 270, and moves the applicator 210 to various locations in a space within a range to be able to adjust the location of the applicator 210. The location of the applicator 210 means not only a position of the applicator 210 but also a direction of the applicator 210. The location adjusting unit 260 moves the applicator 210 along various directions and/or positions like a robot arm.

The control unit 270 controls the components of the ultrasound treatment unit 110. The control unit 270 controls the transducer 220 to radiate ultrasound to a subject for treatment, and controls the diagnostic probe 230 to receive an echo signal reflected from a tissue including the subject. In addition, the control unit 270 transmits an image capturing command signal to the image capturing unit 250 to control the image capturing unit 250 to capture images of skin of the patient 200 with the coupling guiding line 205. Further, the control unit 270 controls the location adjusting unit 260 so that the applicator 210 is located at the coupling region where the coupling guiding line 205 is marked. In order to control the location adjusting unit 260, the control unit 270 receives information of a target location of the applicator 210 from the computer 120. The control unit 270 receives control information of the ultrasound treatment unit 110 from the computer 120.

The computer 120 sets a target location to locate the applicator 210 on the coupling region based on the imaged coupling guiding line 205 and a current location of the applicator 210. In more detail, the computer 120 receives, from the image capturing unit 250, image data of skin of the patient 200 with the coupling guiding line 205, and produces an image including the coupling guiding line 205 based on the received data. The computer 120 calculates a distance from the center 207 of the imaged coupling guiding line 205 to a center 215 of the applicator 210, and an angle made by a line perpendicular to the coupling guiding line 205 at the center 207 thereof and a line perpendicular to the applicator 210 at the center 215 thereof. The computer 120 sets the target location to locate the application 210 on the coupling region based on the calculated distance and angle.

In addition, the computer 120 produces an ultrasound image of a tissue including a subject based on an echo signal received by the diagnostic probe 230. By using the ultrasound image produced in this way, it is possible to obtain a fine adjustment to be used to finely adjust an adjusted location of the applicator 210 that moved to the target location. The adjusted location of the application 210 may be finely adjusted to improve an accuracy in radiating ultrasound and to avoid necrotizing healthy tissues around an area where the ultrasound is radiated. In more detail, the computer 120 may obtain the fine adjustment to be used to finely adjust the adjusted location of the application 210 based on the ultrasound image produced based on the echo signal received by the diagnostic probe 230 at the location of the applicator 210 that is adjusted by the control unit 270 based on the set target location, and a pre-obtained diagnostic image of the tissue including the subject.

Prior to radiating ultrasound, the computer 120 determines whether the applicator 210 is located within a predetermined distance from the coupling region based on the adjusted location of the applicator 210 and the coupling guiding line 205 imaged at the adjusted location. Thus, when a location of the coupling region where the applicator 210 is placed is changed due to motion of the patient 200 after the location of the applicator 210 is adjusted to the set target location, it is possible to radiate the ultrasound after identifying whether the applicator is within the predetermined distance. For example, the predetermined distance may be a maximum distance at which necrosis of a tissue does not exceed a predetermined threshold when ultrasound is radiated. The control unit 270 controls the transducer 220 to radiate ultrasound when the applicator 210 is determined to be located within the predetermined distance from the coupling region.

In addition, subsequent to radiating ultrasound, the computer 120 re-determines whether the applicator 210 is located within the predetermined distance from the coupling region based on the adjusted location of the applicator and the coupling guiding line 205 imaged at the adjusted location. This determination is performed in order to radiate ultrasound after identifying whether the applicator 210 is still within the predetermined distance if the location of the coupling region to which the applicator 210 is placed is changed due to the motion of the patient 200 during ultrasound treatment. The control unit 270 controls the transducer 220 to radiate ultrasound when the applicator 210 is re-determined to be located with the predetermined distance from the coupling region, and the ultrasound treatment is not completed.

The control unit 270 controls the image capturing unit 250, the computer 120, the location adjusting unit 260, and the transducer 220, to locate the applicator 210 at the same coupling region and radiate ultrasound, if the applicator is determined or re-determined to be not located within the predetermined distance. By controlling the diagnostic probe 230, the control unit 270 enables the computer 120 to obtain an ultrasound image, compare the obtained image with a pre-obtained diagnostic image of the tissue including the subject, and obtain a fine adjustment to be used finely adjust the adjusted location of the application 210. The control unit 270 and the computer 120 may be implemented separately as shown in FIG. 2, or may be implemented as a single device or computer.

The display device 130 displays an image including the coupling guiding line 205 imaged by the image capturing unit 250, and/or an ultrasound image of a tissue including a subject that is generated by the computer 120 based on an echo signal received by the diagnostic probe 230. In addition, the display device 130 may display a pre-obtained diagnostic image of the tissue including the subject.

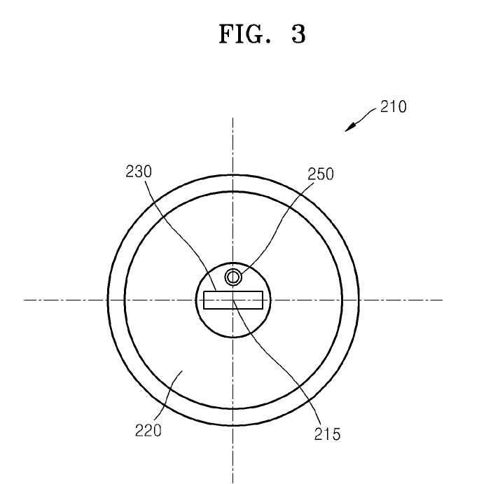

FIG. 3 is view illustrating an example of applicator 210 including the image capturing unit 250 of the ultrasound treatment unit 110 of FIG. 2. In more detail, FIG. 3 is a view seen from a side of the membrane 240 of the applicator 210 that is in contact with skin of a patient. The applicator 210 includes the transducer 220, and the diagnostic probe 230 and the image capturing unit 250 are installed near a center of the transducer 220 and the center 215 of the applicator 210. As shown in FIG. 3, if the diagnostic probe 230 is integrated in the applicator 210, the image capturing unit 250 is located near the center 215 of the applicator 210 by being combined with the diagnostic probe 230.

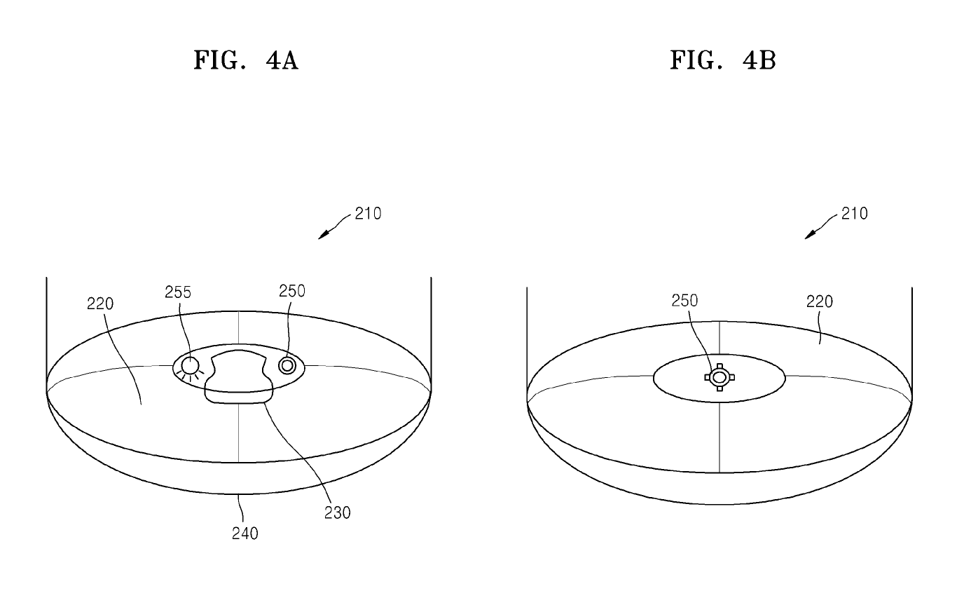

FIGS. 4A and 4B are views illustrating examples of the applicator 210 including the image capturing unit 250 of the ultrasound treatment unit 110 of FIG. 2. One of ordinary skill in the art can understand that other components than those shown in FIGS. 4A and 4B may be additionally included in the applicator 210. The applicator 210 shown in FIGS. 4A and 4B has a cylinder shape, and includes the transducer 220 having a parabolic shape. However, the shapes of the applicator 210 and the transducer 220 are not limited to the above description. If HIFU is radiated from the transducer 220, a cone-beam type ultrasound beam may be formed. Referring to FIG. 4A, the applicator 210 includes the transducer 220, the diagnostic probe 230, the membrane 240, the image capturing unit 250, and the light-emitting device 255. The membrane 240 is located in an area where ultrasound is radiated, a side of the membrane 240 is coupled to a cooling fluid, and another side thereof toward which the ultrasound is radiated is attached to skin of a patient. The membrane 240 is downward convex, and thus, may be tightly attached to the skin of the patient without a gap therebetween. In addition, since the image capturing unit 250 captures an image of a coupling guiding line through the cooling fluid coupled to the membrane 240, the cooling fluid and the membrane 240 are transparent.

The applicator 210 has a shape that allows the diagnostic probe 230 to be combined at a center of the transducer 220. The transducer 220 has a curved shape to concentrate ultrasound on a subject, such as lesion. The diagnostic probe 230 radiates ultrasound to a tissue including the subject, and is combined with the center of the transducer 220 to be able to receive an echo signal reflected from the tissue. The image capturing unit 250 is installed in the applicator 210 along with the diagnostic probe 230 in order to capture the image of the coupling guiding line marked on the skin of the patient 200. In addition, the light-emitting device 255 that operates according to an operation of the image capturing unit 250 is installed in the applicator 210. The image capturing unit 250 and the light-emitting device 255 as shown in FIG. 4A are physically separated, but the image capturing unit 250 may be combined with the light-emitting device 255.

Referring to FIG. 4B, the applicator 210 of the FIG. 4B has a similar configuration to the applicator 210 of FIG. 4A, but a diagnostic probe is not installed in the applicator 210 of FIG. 4B. Descriptions of the same components made with respect to FIG. 4A are omitted with respect to FIG. 4B for conciseness. In FIG. 4A, the diagnostic probe 230 is combined with the transducer 220 at the center thereof, however, in FIG. 4B, the diagnostic probe is separated from the applicator 210 because the diagnostic probe may be used as a separated unit. In FIG. 4B, the image capturing unit 250 is combined with the transducer 220 at the center thereof, and the image capturing unit 250 includes a light-emitting device. That is, the image capturing unit 250 may be installed at any place in the applicator 210. However, if the diagnostic probe 230 is installed in the applicator 210 as shown in FIG. 4A, the image capturing unit 250 is installed near the diagnostic probe 230, and if the diagnostic probe is not installed in the applicator 210 as shown in FIG. 4B, the image capturing unit 250 is installed at the center of the transducer 220.

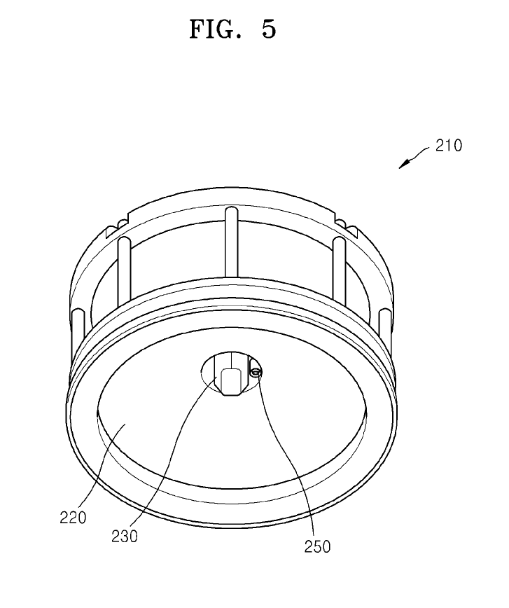

FIG. 5 is a perspective view illustrating another example of the applicator 210 including the image capturing unit 250 of the ultrasound treatment unit 110 of FIG. 2. One of ordinary skill in the art can understand that other components than those shown in FIG. 5 may be additionally included in the applicator 210.

Referring to FIG. 5, the applicator 210 further includes the transducer 220 having a curved shape, and the diagnostic probe 230 located at a center of the transducer 220. The image capturing unit 250 is combined with the diagnostic probe 230 at a side thereof. The image capturing unit 250 may include a light-emitting device. Directions of ultrasound radiated from the transducer 220, directions of ultrasound radiated from the diagnostic probe 230, and a direction of acquiring an image by the image capturing unit 250 are the same. Unlike the examples of the applicator 210 shown in FIGS. 4A and 4B, in FIG. 5, the image capturing unit 250 with the light-emitting device is combined with the diagnostic probe 230 at the side thereof, and is installed in the applicator 210 along with the diagnostic probe 230.

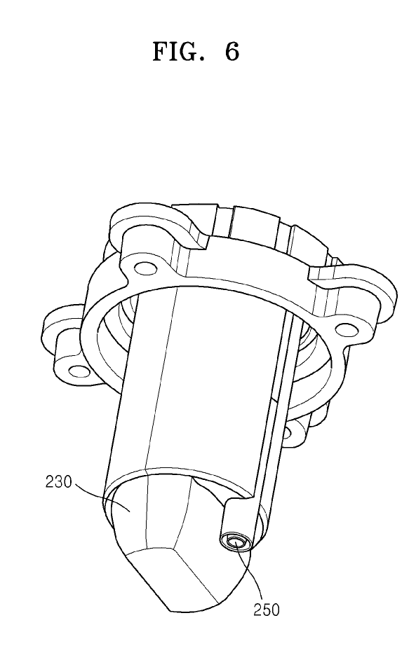

FIG. 6 is a perspective view of the diagnostic probe 230 and the image capturing unit 250 installed in the applicator 210 of FIG. 5. Specifically, FIG. 6 is the perspective view of the diagnostic probe 230 installed in the applicator 210 of FIG. 5 and the image capturing unit 250 combined with the diagnostic probe 230 separately from the applicator 210.

Referring to FIG. 6, the image capturing unit 250 includes a light-emitting device, and is combined with the diagnostic probe 230 via a recess therein. The diagnostic probe 230 is combined with the image capturing unit 250, and has a cylindrical structure so that the diagnostic probe 230 may tightly engage with the transducer 220 at a center thereof in the applicator 210 of FIG. 5, without any gap therebetween. However, the diagnostic probe 230 may have another shape as long as it may be tightly installed without any gap at the center of the transducer 220.

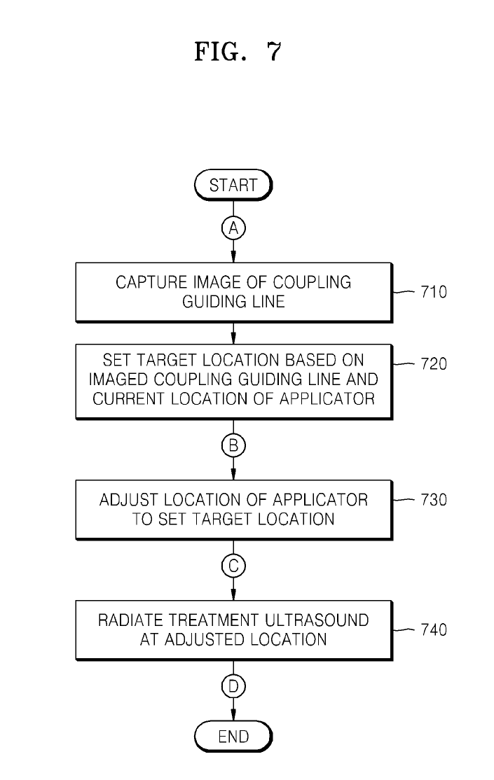

FIG. 7 is a flowchart illustrating an example of an ultrasound treatment method. Descriptions of the ultrasound treatment apparatus 100 of FIGS. 1 and 2 may also apply to the ultrasound treatment method even though they are omitted with respect to FIG. 7 for conciseness.

In operation 710, the image capturing unit 250 of FIG. 2 captures an image of the coupling guiding line 205 that represents a coupling region where the applicator 210 is attached to skin (or a surface) of the patient 200. The coupling guiding line 205 has a different color from a color of the skin of the patient 200, and is a kind of mark information marked on the skin of the patient 200. After a medical practitioner identifies a location of a subject, such as a lesion, on a body of the patient 200, the coupling guiding line 205 is marked on a proper part of the skin of the patient 200 before providing ultrasound treatment. The image capturing unit 250 captures the image including the coupling guiding line 205 that is used to set a target location of the applicator 210 so that the applicator 210 may be coupled to the coupling region represented by the coupling guiding line 205.

In operation 720, the computer 120 of FIG. 1 sets the target location to locate the applicator 210 on the coupling region, based on the imaged coupling guiding line 205 and a current location of the applicator 210. The computer 120 receives data of the image including the coupling guiding line 205 that is imaged by the image capturing unit 250 and information of the current location of the applicator 210, and the computer 120 sets the target location to locate the applicator 210 at the coupling region, based on the received data and information. For example, the computer 120 may receive the data of the image taken from the skin of the patient 200 with the coupling guiding line 205 from the image capturing unit 250, and produce an image including the coupling guiding line 205 based on the received data. In this example, the computer 120 may calculate a distance from the center 207 of the imaged coupling guiding line 205 to the center 215 of the applicator 210, and an angle made by a line perpendicular to the coupling guiding line 205 at the center 207 thereof and a line perpendicular to the applicator 210 at the center 215 thereof. The computer 120 may set the target location of the applicator 210 based on the calculated distance and angle.

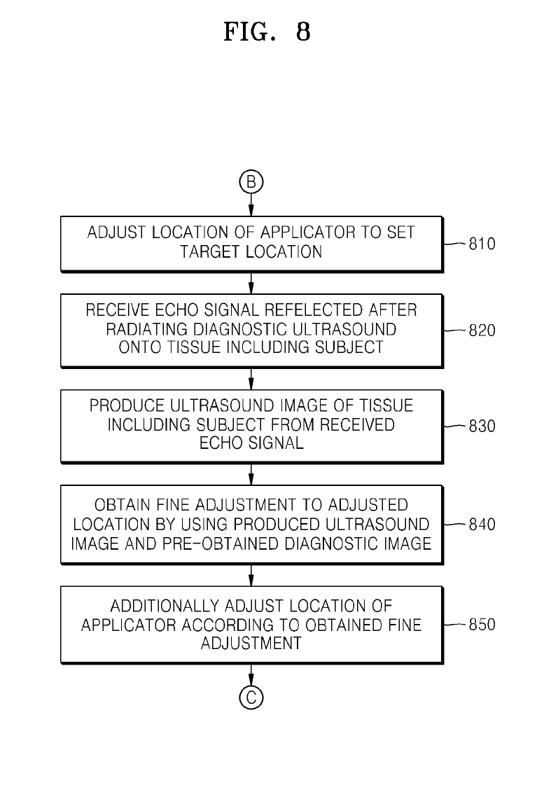

In operation 730, the control unit 270 controls the location adjusting unit 260 to adjust a location of the applicator 210 to the target location set by the computer 120. Descriptions about this operation are made in detail with reference to FIG. 8.

FIG. 8 is a flowchart illustrating an example of an operation of adjusting a location of an applicator. In more detail, FIG. 8 further includes additionally adjusting the location of the applicator 210 of FIG. 2 based on an ultrasound image produced from an echo signal received by the diagnostic probe 230 that radiates diagnostic ultrasound, subsequent to adjusting the location of the applicator 210 to a target location set by the computer 120 of FIG. 1.

In operation 810, the control unit 270 controls the location adjusting unit 260 to adjust the location of the applicator 210 to the target location set by the computer 120. In order to more accurately couple the applicator 210 to a coupling region marked by the coupling guiding line 205, operations 820 to 850 to additionally adjust the location of the applicator 210 may be further performed. Since an accuracy in radiating ultrasound is needed to not necrotize healthy tissues during ultrasound treatment, the additional adjusting to finely adjust the location of the applicator 210 may be performed.

In operation 820, the diagnostic probe 230 radiates diagnostic ultrasound onto a tissue including a subject, and receives an echo signal reflected from the tissue, after the location of the applicator 210 is adjusted to the target location set by the computer 120.

In operation 830, the computer 120 produces an ultrasound image of the tissue including the subject from the echo signal received by and from the diagnostic probe 230.

In operation 840, the computer 120 obtains a fine adjustment to the adjusted location by using a pre-obtained diagnostic image of the tissue including the subject and the ultrasound image produced from the echo signal received by the diagnostic probe 230. The pre-obtained diagnostic image may be an ultrasound image pre-obtained at a location when the coupling guiding line 205 is marked on the skin of the patient 200. The fine adjustment is a value used to additionally adjust the location of the applicator 210 to be able to radiate ultrasound to the same subject with reference to images of tissues of a body of the patient 200 at the target location set based on the coupling guiding line 205 marked on the skin of the patient 200.

In operation 850, the control unit 270 controls the location adjusting unit 260 to additionally adjust the location of the applicator 210 according to the fine adjustment obtained by the computer 120.

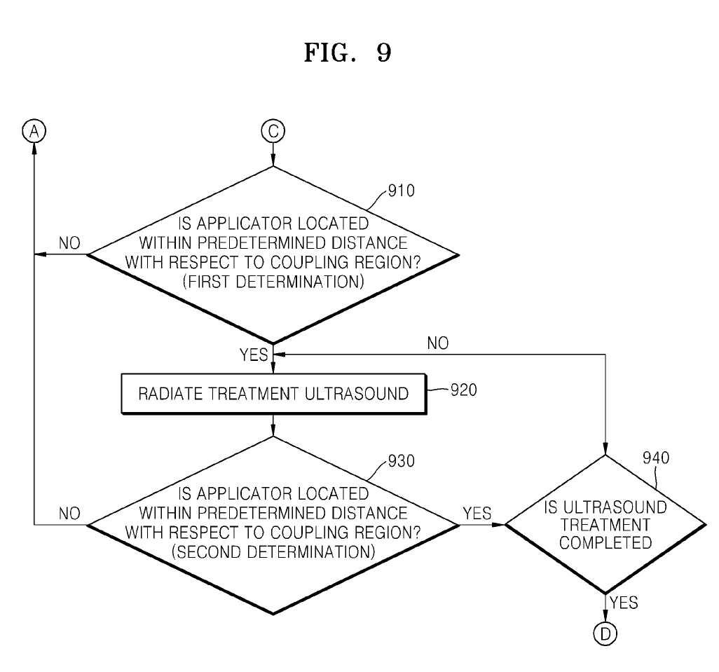

Referring back to FIG. 7, in operation 740, the control unit 270 controls the transducer 220 to radiate treatment ultrasound at the location of the applicator that is adjusted by the location adjusting unit 260. The adjusted location of the applicator 210 may be the target location set based on the coupling guiding line 205 or a location finely adjusted to the set target location. The operation of the radiating of the ultrasound will be described in detail with reference to FIG. 9.

FIG. 9 is a flowchart illustrating an example of an operation of radiating ultrasound from an applicator at an adjusted location. In operation 910, prior to radiating treatment ultrasound, the computer 120 of FIG. 1 makes a first determination on whether the applicator 210 of FIG. 2 is located within a predetermined distance with respect to a coupling region marked by the coupling guiding line 205 based on an adjusted location of the applicator 210 and the coupling guiding line 205 imaged at the adjusted location. For example, the predetermined distance may be a maximum distance where necrosis of a healthy tissue does not exceed a predetermined threshold when the ultrasound is radiated.

If the applicator 210 is determined to be not located within the predetermined distance, the operations 710 to 730 of FIG. 7 are repeated to re-adjust the location of the applicator 210. On the contrary, if the applicator 210 is determined to be located within the predetermined distance, operation 920 is performed.

In the operation 920, the control unit 270 controls the transducer 220 so that the transducer 220 radiates the treatment ultrasound.

In operation 930, the computer 120 makes a second determination on whether the applicator 210 is located within the predetermined distance with respect to the coupling region based on the adjusted location of the applicator 210 and the coupling guiding line 205 imaged at the adjusted location. This is to determine whether the applicator 210 is not in contact with the skin of the patient 200, or whether there is a change in a location where the applicator 210 is to contact the skin of the patient 200 due to motion of the patient 200, after the applicator 210 is placed in contact with the skin of the patient 200 at the adjusted location and radiates the ultrasound.

If the applicator 210 is determined to be not located within the predetermined distance, the operations 710 to 730 of FIG. 7 are repeated to re-adjust the location of the applicator 210. On the contrary, if the applicator 210 is determined to be located within the predetermined distance, operation 940 is performed.

In the operation 940, the control unit 270 determines whether the ultrasound treatment is completed. If the ultrasound is determined to be not completed, the operation 920 is re-performed, and the control unit 270 controls the transducer 220 so that the transducer 220 radiates the treatment ultrasound again. On the contrary, if the ultrasound treatment is determined to be completed, the operation of radiating the ultrasound ends. The control unit 270 may determine whether the ultrasound treatment is completed by receiving a completion signal from the computer 120. The computer 120 may receive a command to stop the ultrasound treatment from a user, and/or may send, to the control unit 270, a signal indicating that the ultrasound treatment is completed if a time previously-entered in the computer 120 (e.g., by the user) has passed.

The examples of an ultrasound treatment apparatus and an ultrasound treatment method described above may identify a location of a coupling region where an applicator is placed by using only an image capturing unit, without using a marker unit and electronic devices that track the marker unit that are attached to a patient's skin. Thus, it may be easy to install the apparatus and manufacture the same at low costs. In addition, there may be no need to consider interference due to ultrasound energy transfer by the marker unit attached to the patient's skin. Further, it may be possible to enhance an accuracy of radiating ultrasound to a subject, such as a lesion, by contacting the applicator on the patient's skin at the same location of the coupling region.

The various units, elements, and methods described above may be implemented using one or more hardware components, one or more software components, or a combination of one or more hardware components and one or more software components.

A hardware component may be, for example, a physical device that physically performs one or more operations, but is not limited thereto. Examples of hardware components include microphones, amplifiers, low-pass filters, high-pass filters, band-pass filters, analog-to-digital converters, digital-to-analog converters, and processing devices.

A software component may be implemented, for example, by a processing device controlled by software or instructions to perform one or more operations, but is not limited thereto. A computer, controller, or other control device may cause the processing device to run the software or execute the instructions. One software component may be implemented by one processing device, or two or more software components may be implemented by one processing device, or one software component may be implemented by two or more processing devices, or two or more software components may be implemented by two or more processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

A processing device configured to implement a software component to perform an operation A may include a processor programmed to run software or execute instructions to control the processor to perform operation A. In addition, a processing device configured to implement a software component to perform an operation A, an operation B, and an operation C may have various configurations, such as, for example, a processor configured to implement a software component to perform operations A, B, and C; a first processor configured to implement a software component to perform operation A, and a second processor configured to implement a software component to perform operations B and C; a first processor configured to implement a software component to perform operations A and B, and a second processor configured to implement a software component to perform operation C; a first processor configured to implement a software component to perform operation A, a second processor configured to implement a software component to perform operation B, and a third processor configured to implement a software component to perform operation C; a first processor configured to implement a software component to perform operations A, B, and C, and a second processor configured to implement a software component to perform operations A, B, and C, or any other configuration of one or more processors each implementing one or more of operations A, B, and C. Although these examples refer to three operations A, B, C, the number of operations that may implemented is not limited to three, but may be any number of operations required to achieve a desired result or perform a desired task.

Software or instructions for controlling a processing device to implement a software component may include a computer program, a piece of code, an instruction, or some combination thereof, for independently or collectively instructing or configuring the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments for implementing the examples disclosed herein can be easily constructed by a programmer skilled in the art to which the examples pertain based on the drawings and their corresponding descriptions as provided herein.

A computing system or a computer may include a microprocessor that is electrically connected to a bus, a user interface, and a memory controller, and may further include a flash memory device. The flash memory device may store N-bit data via the memory controller. The N-bit data may be data that has been processed and/or is to be processed by the microprocessor, and N may be an integer equal to or greater than 1. If the computing system or computer is a mobile device, a battery may be provided to supply power to operate the computing system or computer. It will be apparent to one of ordinary skill in the art that the computing system or computer may further include an application chipset, a camera image processor, a mobile Dynamic Random Access Memory (DRAM), and any other device known to one of ordinary skill in the art to be included in a computing system or computer. The memory controller and the flash memory device may constitute a solid-state drive or disk (SSD) that uses a non-volatile memory to store data.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An apparatus configured to provide ultrasound treatment, comprising:
an applicator configured to radiate ultrasound to a subject;
an image capturing unit configured to capture an image of a coupling guiding line on a patient where the applicator is to be placed;
a computer configured to set a target location of the applicator based on the image and a current location of the applicator;
a location adjusting unit configured to adjust a location of the applicator; and
a control unit configured to control the location adjusting unit to adjust the location of the applicator to the target location,
wherein the applicator is configured to radiate ultrasound at the adjusted location.

2. The apparatus of claim 1, wherein the image capturing unit is installed in the applicator.

3. The apparatus of claim 1, wherein the image capturing unit comprises a light-emitting device.

4. The apparatus of claim 1, wherein the computer is configured to:
calculate a distance from a center of the coupling guiding line in the image to a center of the applicator, and an angle between a line perpendicular to the coupling guiding line at the center thereof and a line perpendicular to the applicator at the center thereof; and
set the target location based on the distance and the angle.

5. The apparatus of claim 1, further comprising:
a diagnostic probe installed in the applicator, and configured to
radiate ultrasound to a tissue comprising the subject at the adjusted location, and
receive an echo signal reflected from the tissue.

6. The apparatus of claim 5, wherein the computer is further configured to:
produce an ultrasound image of the tissue based on the echo signal; and
obtain a fine adjustment of the adjusted location based on a pre-obtained diagnostic image of the tissue comprising the subject and the ultrasound image.

7. The apparatus of claim 6, wherein the control unit is further configured to:
control the location adjusting unit to adjust the adjusted location based on the fine adjustment.

8. The apparatus of claim 6, further comprising:
a display device configured to display the image of the coupling guiding line and/or the ultrasound image.

9. The apparatus of claim 1, wherein:
the computer is configured to determine whether the applicator is within a predetermined distance from the coupling guiding line based on the adjusted location and the coupling guiding line imaged at the adjusted location; and
the control unit is configured to control the applicator to radiate the ultrasound at the adjusted location in response to the applicator being determined to be within the predetermined distance.

10. The apparatus of claim 9, wherein:
the computer is further configured to
re-determine whether the applicator is within the predetermined distance from the coupling guiding line based on the adjusted location and the coupling guiding line imaged at the adjusted location, in response to the applicator being controlled to radiate the ultrasound, and
determine whether the ultrasound treatment is completed in response to the applicator being re-determined to be within the predetermined distance; and
the control unit is further configured to re-control the applicator to radiate the ultrasound at the adjusted location in response to the ultrasound treatment being determined to be not completed.

11. The apparatus of claim 10, wherein the control unit is further configured to control the image capturing unit, the computer, and the location adjusting unit, to locate the applicator at the coupling guiding line, in response to the applicator being determined or re-determined to be not within the predetermined distance.

12. A method of providing ultrasound treatment, the method comprising:
capturing an image of a coupling guiding line on a patient where an applicator is to be placed, the applicator configured to radiate ultrasound to a subject;
setting a target location of the applicator based on the image and a current location of the applicator;
adjusting a location of the applicator to the target location; and
radiating the ultrasound at the adjusted location.

13. The method of claim 12, wherein the setting of the target location comprises:
calculating a distance from a center of the coupling guiding line in the image to a center of the applicator, and an angle between a line perpendicular to the coupling guiding line at the center thereof and a line perpendicular to the applicator at the center thereof; and
setting the target location based on the distance and the angle.

14. The method of claim 12, further comprising:
radiating ultrasound to a tissue comprising the subject at the adjusted location;
receiving an echo signal reflected from the tissue;
producing an ultrasound image of the tissue based on the echo signal;
obtaining a fine adjustment of the adjusted location based on a pre-obtained diagnostic image of the tissue comprising the subject and the ultrasound image; and
adjusting the adjusted location based on the fine adjustment.

15. The method of claim 12, wherein the radiating of the ultrasound comprises:
determining whether the applicator is within a predetermined distance from the coupling guiding line based on the adjusted location and the coupling guiding line imaged at the adjusted location; and
radiating the ultrasound at the adjusted location in response to the applicator being determined to be within the predetermined distance.

16. The method of claim 15, wherein the capturing of the image, the setting of the target location, and the adjusting of the location of the applicator are repeated, in response to the applicator being determined to be not within the predetermined distance.

17. The method of claim 12, further comprising:
determining whether the applicator is within a predetermined distance from the coupling guiding line based on the adjusted location and the coupling guiding line imaged at the adjusted location, in response to the radiating of the ultrasound;
determining whether the ultrasound treatment is completed in response to the applicator being determined to be within the predetermined distance; and
re-radiating the ultrasound at the adjusted location in response to the ultrasound treatment being determined to be not completed.

18. The method of claim 17, wherein the capturing of the image, the setting of the target location, and the adjusting of the location of the applicator are repeated, in response to the applicator being determined to be not within the predetermined distance.

19. The method of claim 12, wherein the coupling guiding line has a color that is different from a color of skin of the patient, and is marked on the skin.

20. A non-transitory computer-readable storage medium storing a program comprising instructions to cause a computer to execute the method of claim 12.

21. An apparatus comprising:
an applicator configured to radiate ultrasound to a patient;
an image capturing unit configured to capture an image of a mark on the patient where the applicator is to be placed; and
a computer configured to set a target location of the applicator based on the image.

22. The apparatus of claim 21, further comprising:
a location adjusting unit configured to adjust a location of the applicator,
wherein the computer is further configured to
control the location adjusting unit to adjust the location of the applicator to the target location, and
control the applicator to radiate ultrasound at the adjusted location.
